(19) United States
(12) Patent Application Publication
(10) Pub. No.: US 2026/0345102 A1
(43) Pub. Date: Nov. 5, 2026

(54) METHOD FOR CONDUCTING AN IMAGING EXAMINATION

(71) Applicant: Siemens Healthineers AG, Forchheim (DE)

(72) Inventor: Ulrich BATZER, Spardorf (DE)

(73) Assignee: Siemens Healthineers AG, Forchheim (DE)

(21) Appl. No.: 19/661,849

(22) Filed: Apr. 29, 2026

(30) Foreign Application Priority Data
Apr. 30, 2025 (DE) 10 2025 116 654.4

(51) Int. Cl. A61B 6/46; A61B 5/055

(52) U.S. Cl. CPC A61B 6/46; A61B 5/055

(57) ABSTRACT

The method according to one or more example embodiments for conducting an imaging examination of a patient via a medical imaging system includes extracting speech sounds of the patient and system sounds of an imaging system from an audio input signal, modifying speech sounds and/or weighted system sounds; combining the possibly modified speech sounds and the possibly modified system sounds into an audio output signal via the computer unit; and providing, via a reproduction unit, to a user of the imaging system for monitoring the imaging examination the combined audio output signal while the imaging examination is being conducted.

### CROSS-REFERENCE TO RELATED APPLICATION(S)

The present application claims priority under 35 U.S.C. § 119 to German Patent Application No. 10 2025 116 654.4, filed Apr. 30, 2025, the entire contents of which is incorporated herein by reference.

### FIELD

One or more example embodiments relates to a method for conducting an imaging examination of a patient via a medical imaging system, such as a CT, MRI or C-arm imaging system, to the imaging system, and to an associated computer program product.

### RELATED ART

A conventional imaging system typically produces numerous system sounds while operating. Sounds from different components of the imaging system typically intermix. Sounds from equipment that is not part of the imaging system can also intermix with and/or be superimposed on the system sounds.

A user of the imaging system, for example the operator, in particular a radiographer and/or a radiologist, is normally located during operation of the imaging system in a control room, which is spatially separate from the examination room containing the imaging system. The control room and the examination room may be shielded from each other in particular against X-rays and/or electromagnetic fields. In the case of X-rays, this can serve in particular to protect the user from a potential hazard posed by the X-rays. In the case of an MRI imaging system, the magnetic field of the imaging system is typically shielded to avoid impairing the functionality of technical equipment outside the examination room.

As a result of the spatial separation, the patient being examined by the imaging system is not in the immediate vicinity, in particular not within hearing range, of the user of the imaging system. Therefore, according to the prior art, a voice communication facility is usually installed that allows voice communication between the patient and the user. For this purpose, the voice communication facility typically has a microphone and a loudspeaker for both the patient and the user.

In particular, the system sounds often make it difficult for the user to understand the patient. It is known to use a filter and/or a frequency-based means to improve the audio signal comprising in particular the speech sounds of the patient and, as unwanted sound, the system sounds of the imaging system. Alternatively or additionally, in particular a trained machine learning model can be used to improve the audio signal. The improving of the audio signal usually serves to accentuate the speech sounds over the system sounds, in particular to the level that the latter are no longer perceptible at all to the user.

### SUMMARY

One or more example embodiments provides a method for conducting an imaging examination of a patient via a medical imaging system, such as a CT or MRI imaging system, the imaging system, and an associated computer program product which are improved, and in particular ensure improved performance of the imaging examination, such as through improved monitoring of the imaging examination.

### BRIEF DESCRIPTION OF THE DRAWINGS

The invention is described and explained in greater detail below with reference to the exemplary embodiments shown in the figures, where the same reference signs are generally used in the following description of the figures to denote structures and units that remain substantially the same as in the first appearance of the structure or unit concerned, and in which:

FIG. 1 shows a method according to one or more example embodiments;

FIG. 2 shows a first exemplary embodiment of the method; and

FIG. 3 shows an imaging system according to one or more example embodiments.

### DETAILED DESCRIPTION

The method according to one or more example embodiments for conducting an imaging examination of a patient via a medical imaging system, in particular a CT or MRI or C-arm imaging system, comprises the steps:
starting the imaging examination via the imaging system;
capturing an audio input signal via at least one microphone, wherein the at least one microphone is set up and oriented so as to detect, and provide as the audio input signal, sounds of the patient and of the imaging system while the imaging examination is being conducted;
extracting speech sounds of the patient and system sounds of the imaging system from the audio input signal via a computer unit;
classifying the system sounds via the computer unit;
weighting, via the computer unit, the classified system sounds with at least two different weighting factors retrieved from a memory unit;
determining a modification instruction, which depends on the patient of the imaging examination, by analyzing the perceptibility of the extracted speech sounds via the computer unit;
modifying the speech sounds and/or the weighted system sounds in accordance with the modification instruction via the computer unit;
combining the possibly modified speech sounds and the possibly modified system sounds into an audio output signal via the computer unit;
providing, via a reproduction unit, to a user of the imaging system for monitoring the imaging examination the combined audio output signal while the imaging examination is being conducted.

The imaging system according to one or more example embodiments for carrying out the method according to one or more example embodiments has
an imaging unit;
the computer unit;
the reproduction unit; and
the at least one microphone.

Reference can be made to all the statements relating to the method according to one or more example embodiments.

The computer program product according to one or more example embodiments comprises program code means which, when the program is executed by a computing unit, cause said computing unit to carry out the method according to one or more example embodiments.

Reference can be made to all the statements relating to the method according to one or more example embodiments and to the imaging system according to one or more example embodiments.

Features, advantages or alternative embodiments can be applied to the other claimed subject matter in each case, and vice versa. In other words, claims and embodiments of the imaging systems can be improved by features described or claimed in the context of the respective methods. In this case, the functional features of the methods are implemented by physical units, in particular components, of the imaging system.

Claims and embodiments for providing a trained machine learning model can be improved by features described or claimed in the context of conducting an imaging examination. In particular, datasets used in the methods and imaging systems for conducting an imaging examination can have the same attributes and features as the corresponding datasets in the methods and imaging systems for providing a trained machine learning model, and the trained machine learning models provided by the respective methods and imaging systems can be used in the methods and imaging systems for conducting an imaging examination.

According to one or more example embodiments, the system sounds give the user of the imaging system an indication of the extent to which the imaging examination is running at all or in particular according to plan. In particular, at least some of the system sounds contain information that can assist the user in conducting the imaging examination. The monitoring of the system sounds is at least a facility and/or a constituent part of monitoring the conducting of the imaging examination.

One or more example embodiments in particular offers the advantage that, in contrast with the known prior art, system sounds are not partially or fully suppressed but are classified and weighted according to their type. Depending on weighting, the weighted system sounds are, via the reproduction unit, perceptible to the user in addition to the speech sounds. This advantageously allows the user to monitor the imaging examination, thereby improving the monitoring of the imaging examination and the imaging examination itself.

The imaging examination is in particular a method for determining medical images of a patient. Depending on the type of the imaging system, the determined images of the patient are 2D or 3D or 4D image datasets.

The medical imaging system can be in particular a CT imaging system, an MRI imaging system or a C-arm imaging system. The CT imaging system usually comprises a CT gantry as part of the imaging unit, having a part that rotates with respect to a stationary part, or a displaceable part. Purely stationary CT imaging systems are also known. The CT imaging system uses in particular an X-ray source as part of the imaging unit for determining the medical images. The MRI imaging system usually comprises a plurality of MRI coils as part of the imaging unit, in particular a main magnetic field coil and a gradient coil. The MRI imaging system typically uses the MRI coils for determining the medical images.

Alternatively, the medical imaging system can be a C-arm imaging system. The C-arm imaging system comprises in particular a movable C-arm as part of the imaging unit, on which is arranged, inter alia, an X-ray source as part of the imaging unit for determining the medical images.

The starting of the imaging examination is intended in the broad sense here. The starting refers in particular to that point in time at which one of the activities mentioned below is first begun as part of the imaging examination of the patient. For example, the starting of the imaging examination can coincide with preparing the patient for the imaging examination and/or with actuating a component of the imaging system, whereby is achieved in particular an interaction in preparing and/or conducting the imaging examination with the patient. The imaging examination via the imaging system can be started in particular manually by the user of the imaging system and/or automatically.

For example, the preparing of the patient may comprise giving explanations to the patient outside or inside the examination room and/or at least before the patient is placed on a patient couch. The preparing of the patient may comprise positioning the patient couch, on which the patient is supported, relative to the imaging system. The positioning of the patient couch can comprise in particular a movement, preferably inward travel, of the patient couch into the examination region of the imaging system. Alternatively or additionally, the preparing of the patient can comprise placing venous access for injecting contrast agent.

The actuating of the component of the imaging system can comprise triggering the movement of a moving part of the imaging system. The moving part may be the patient couch, the C-arm, a rotating part of the gantry, and/or a displaceable part of the gantry. The triggering of the movement of the moving part can be suitable for orientating the imaging system, in particular the moving part of the imaging system, relative to the patient.

Alternatively or additionally, the actuating of the component of the imaging system can comprise triggering an image determination routine via one, a plurality of, or all the components of the imaging system, with the images of the patient being determined via the image determination routine. The image determination routine can comprise instructions for generating X-rays via the X-ray source, for emitting RF pulses via an MRI coil, for outputting patient instructions to the patient via a loudspeaker, for example commands to hold one's breath or keep still, for capturing the patient's vital parameters via a sensor, and/or for injecting contrast agent into the patient via a contrast-agent injection pump.

The time period during the imaging examination begins by definition with the starting of the imaging examination and ideally ends at the earliest with the conclusion of the image determination routine. It is conceivable that the time period during the imaging examination does not end until the end of the image determination routine or post-processing of the imaging examination. The post-processing of the imaging examination can comprise in particular reconstructing the images and/or moving the patient couch out of the imaging system.

The capture of the audio input signal can take place over the entire time period during the imaging examination or alternatively for at least one contiguous partial time period that lies within the time period during the imaging examination. The at least one contiguous partial time period can be substantially shorter than the entire time period during the imaging examination. Contiguous means in particular uninterrupted.

Advantageously, all the steps from capturing the audio input signal up to and including providing the combined audio output signal take place in real time, in principle continuously and/or repeatedly in succession and/or in parallel. This is illustrated in the following paragraph by way of example for these steps.

The capture of the audio input signal is performed in particular with negligible delay, preferably in real time. The capture of the audio input signal can be performed in principle continuously and/or repeatedly in succession and/or in parallel. The same applies in particular to the detecting of the sounds and/or the providing of the audio input signal.

It follows from the two previous paragraphs that the time of playback of a sound corresponds substantially to the time of detection of the sound, because the steps from capturing the audio input signal up to and including providing the combined audio output signal are performed without delay, except for the shift according to an advantageous embodiment below.

Suitable microphones are fundamentally known. The capture of the audio input signal can be performed via a single microphone or a plurality of microphones that can preferably be spatially distributed.

The at least one microphone is in principle designed to detect sounds. For this purpose, the at least one microphone has, for example, a detection interface, for instance a sound sensor. The at least one microphone has in particular one or more detection channels. The at least one microphone, in particular its detection interface, is in particular oriented onto the patient and/or the imaging system.

Sounds of the patient comprise in particular the patient's speech and optionally further speech sounds. In addition, sounds of the patient can comprise noises from the patient and/or body movements, in particular respiratory, leg and/or arm movements. The further speech sounds comprise in particular the speech of a physician and/or a further person.

Sounds of the imaging system comprise in particular sounds that are produced by the imaging system when the imaging examination is being conducted, and optionally further ambient sounds. The sounds of the imaging system are in particular operating sounds of the imaging system while the imaging examination is being conducted. The further ambient sounds can be sounds that are produced by external equipment, which is not part of the imaging system but assists the imaging examination, while the imaging examination is being conducted.

The at least one microphone can comprise a transducer unit for converting the detected sounds into the audio input signal. The at least one microphone can provide the audio input signal at an output interface, for example for transfer to the computer unit. The output interface can be wired or wireless.

The at least one microphone can be attached in particular to the imaging system. A plurality of microphones can be arranged, for example, across the examination room, some on the imaging system and some detached from the imaging system. The at least one microphone can comprise a mixer unit, which mixer unit can be used to combine detected sounds and/or provided audio input signals from the microphone or a plurality of microphones into the audio input signal, which can be transferred to the computing unit, for instance via the output interface.

The extracting of speech sounds of the patient and system sounds of the imaging system from the audio input signal can comprise separating and/or filtering the audio input signal. The extracting is performed in particular in accordance with extraction program code means via the computer unit. The extraction program code means can comprise in particular a first trained machine learning model. The extracting is performed in particular under the condition that detected sounds of the patient shall be assigned to the speech sounds, and detected sounds of the imaging system shall be assigned to the system sounds. It is conceivable that in the extracting, some of those sounds that do not correspond to speech sounds or system sounds are filtered out and discarded.

The classifying of the system sounds comprises in particular assigning the system sounds to the different components of the imaging system and optionally to components of external equipment, which is not part of the imaging system but assists the conducting of the imaging examination. The classifying comprises in particular dividing the system sounds into sound classes. The classifying can be performed in particular by segment and/or by channel. By segment means that the system sounds are divided into a plurality of time segments of arbitrary length, with each of the plurality of segments being classified. By channel means that system sounds from different sound classes are intermixed and/or superimposed in segments, and the classifying involves separating the mixed and/or superimposed system sounds and classifying each of the separated system sounds.

Each component typically has at least one distinct sound class. In principle, a component can be set up to produce different sounds that can each be assigned to a distinct sound class. It is conceivable that in the classifying, the system sounds are assigned solely to one sound class, to a plurality of sound classes or to all the sound classes of components of the imaging system. It is conceivable that the assigned sound classes change by segment and/or sometimes occur simultaneously. Usually, at least some of the system sounds are not assigned to any component of the imaging system but instead are assigned as unwanted background sound, for example to the environment of the imaging system.

In particular, the system sounds comprise sounds from at least two of the sound classes in the following non-exhaustive list, wherein the system sounds are divided during classification into the at least two sound classes:
unwanted background sounds;
patient couch, in particular scan-specific movement of the support part of the patient couch and/or travel of the entire patient couch;
MRI coil, in particular gradient coil;
coolant pump, in particular helium pump;
rotating part of the CT gantry;
displaceable part of the CT gantry;
X-ray source;
contrast-agent injection pump;
patient instruction;
C-arm;
vital-parameter sensor, in particular patient monitoring system; and/or
external equipment.

The classifying of the system sounds is performed in particular after the extracting of the system sounds from the audio input signal. The classifying of the system sounds can be performed in accordance with classification program code means via the computer unit. The classification program code means can comprise in particular a second trained machine learning model and/or the first trained machine learning model.

A weighting factor can be time-resolved in particular. In particular, a weighting factor can vary between classified time segments of the same sound class. A weighting factor can be time-resolved such that a classified time segment of the sound class has a weighting factor that differs from a weighting factor of an earlier or later classified time segment of the same sound class. In particular, time segments of the sounds can be assigned time-resolved weighting factors in accordance with the at least two sound classes. Time-resolved weighting factors are advantageous in particular because time segments of sounds from the same sound class can differ in their relevance and/or criticality to the monitoring of the imaging examination.

In general, the weighting factor reflects the necessity for the user of the imaging system to perceive the correspondingly weighted sound. Typically, a weighting factor is higher when the sound to be provided, in particular of the time segment concerned, of the sound class is more critical and/or relevant to monitoring the imaging examination. The relevance and/or criticality of a sound to monitoring the imaging examination can take into account in particular to what extent the sound in question must be reproduced in time with the detection of same, or whether it can be reproduced advanced or delayed.

The weighting of the classified system sounds can comprise retrieving the weighting factors. Alternatively or additionally, the weighting factors can be retrieved before the weighting. The retrieving of the weighting factors from the memory unit is intended in the broad sense here and can comprise executing program code means in which the weighting factors are hard-programmed and which are buffered in RAM as the memory unit. It is conceivable that the user, before or during the imaging examination, adjusts the weighting factors via an input means, and submits the adjusted weighting factors to the memory unit for weighting the classified system sounds. The memory unit can be part of the computer unit and/or of the imaging system.

The weighting factors are typically each assigned to one sound class. A weighting factor specifies in arbitrary units a relative change in relation to the perceptibility of the system sounds in this sound class. A weighting factor greater than 1 increases the perceptibility of such system sounds. A weighting factor equal to 1 keeps the perceptibility of such system sounds the same, and therefore system sounds assigned to this sound class remain substantially unchanged. A weighting factor less than 1 reduces the perceptibility of such system sounds. A weighting factor equal to zero eliminates the perceptibility of such system sounds. At least one of the weighting factors can equal 1. Weighting factors not equal to 1 alter the perceptibility in particular by a corresponding adjustment to the phase and/or frequency and/or amplitude.

The weighting of the classified system sounds is typically performed after the classifying of the system sounds. The weighting of the classified system sounds can be performed in accordance with weighting program code means via the computer unit.

The determining of the modification instruction comprises in particular the analyzing of the perceptibility of the speech sounds, in particular in comparison with the weighted system sounds. The perceptibility of the speech sounds can be parameterized in perceptibility values, which describe the perceptibility of speech sounds. The determining of the modification instruction is performed in particular under the condition that the speech sounds and the system sounds can be perceived by a human recipient, in particular by the user, when the combined audio output signal is provided, with the speech sounds preferably being given a higher priority than the system sounds.

In particular, the modification instruction specifies that, before being combined, the speech sounds and/or the system sounds are modified via an adjustment to the phase and/or frequency and/or amplitude and/or time of playback. The modifying is intended to be performed in particular in such a way that their essential character is unchanged but, in the event of other sounds occurring close in time, they interfere less with the perception of these and/or are themselves more easily perceived.

The perceptibility values can typically be split into objective invariable values that relate to a volume level for the patient and/or average human hearing thresholds, and subjective variable values that ensure the user can identify the patient's voice and/or ensure the user understands the patient. Unlike the weighting factors, the perceptibility values are typically set not purely objectively but take into account the individual characteristics of the way the patient speaks.

The determining of the modification instruction can comprise retrieving the, in particular objective, perceptibility values. Alternatively or additionally, the, in particular objective, perceptibility values can be retrieved before the determining of the modification instruction. The retrieving of the, in particular objective, perceptibility values from the memory unit is intended in the broad sense here and can comprise executing program code means in which the, in particular objective, perceptibility values are hard-programmed and which are buffered in RAM as the memory unit. It is conceivable that the user, before or during the imaging examination, adjusts the, in particular objective or subjective, perceptibility values via an input means, and submits the adjusted perceptibility values to the memory unit for determining the modification instruction. The memory unit can be part of the computer unit and/or of the imaging system.

The determining of the modification instruction is typically performed after the extracting of the speech sounds. The determining of the modification instruction can be performed via the computer unit in accordance with determination program code means, which depend in particular on the objective perceptibility values and the subjective perceptibility values. The determination program code means can comprise in particular a third trained machine learning model and/or the first trained machine learning model and/or the second trained machine learning model.

The modifying of the speech sounds and/or the weighted system sounds comprises in particular executing the determined modification instruction via the computer unit. Depending on the determined modification instruction, in the modifying are modified only the speech sounds or only the weighted system sounds or both the speech sounds and the weighted system sounds. The modifying can be performed in particular by a corresponding adjustment to the phase and/or frequency and/or amplitude. The modifying is performed in particular under the condition that preferably the intelligibility of the speech sounds over the weighted system sounds is ensured.

The modifying is performed in particular after the determining of the modification instruction and after the weighting of the system sounds. The modifying can be performed in accordance with modification program code means via the computer unit. The modification program code means can comprise in particular a fourth trained machine learning model and/or the first trained machine learning model and/or the second trained machine learning model and/or the third trained machine learning model.

The combining can comprise mixing and/or adding the possibly modified speech sounds and the possibly modified system sounds. The possibly modified speech sounds are those speech sounds that have been modified in the preceding step in accordance with the modification instruction, otherwise the extracted speech sounds of the patient. The possibly modified system sounds are those system sounds that have been modified in the preceding step in accordance with the modification instruction, otherwise the weighted system sounds of the imaging system.

The combining is performed in particular after the modifying. The combining can be performed in accordance with combining program code means via the computer unit. The computer unit can be designed to transfer the combined audio output signal to the reproduction unit and/or the memory unit, and/or to provide it for the reproduction unit.

The providing of the combined audio output signal can comprise receiving and/or retrieving the combined audio output signal from the computer unit and/or the memory unit. The providing of the audio output signal to the user of the imaging system can comprise reproduction of the audio output signal to the user via the reproduction unit.

The reproduction unit can comprise in particular a loudspeaker and/or headphones. The reproduction unit can be part of the voice communication facility that the user can use while the imaging examination is being conducted.

The user will advantageously conduct the monitoring of the imaging examination according to the provided audio output signal. The monitoring of the imaging examination comprises in particular continuing the imaging examination as initially started, adjusting the imaging examination, for example by outputting a command via a loudspeaker to the patient and/or adjusting the instruction in the imaging routine, ending the imaging examination prematurely, and/or triggering an alarm signal.

An embodiment provides that the determined modification instruction comprises a shift in the time of playback of at least one segment of the speech sounds. Preferably, the time of playback can be modified in accordance with the modification instruction such that a time overlap with a segment of the system sounds is reduced. This embodiment is advantageous in particular because superposition of system sounds with speech sounds can be reduced thereby. This embodiment can also be advantageous in the case of forward masking or backward masking of the perceptibility by the user. Forward masking occurs in particular if a loud sound as a masker hampers or prevents a subsequent quieter sound being perceptible, in particular over a time period of up to 1 s, for example 50 to 500 ms, after the end of the masker. Backward masking occurs in particular if a loud sound as a masker hampers or prevents a quieter sound being perceptible that occurs shortly beforehand, for example over a time period of up to 0.5 s, in particular 5 to 20 ms, before the masker occurs. The shift in the time of playback can equal 20 s maximum, in particular less than 2 s, or in particular, given high relevance, less than 1 s, but more than 0 s. The shift in the time of playback means in particular that a time offset with respect to the time of detection and the resultant time of playback of the sound in the form of the shift is determined, which the computer unit adds onto the actual time of playback until the at least one segment of the speech sounds is provided after the shift has elapsed.

A development of the previous embodiment provides that the determining of the modification instruction is performed depending on the time-resolved weighting factor of the classified system sounds. This embodiment is advantageous in particular for avoiding a shift taking place when the particular segment of the system sounds can be superimposed anyway on the speech sounds because it has a comparatively low weighting factor.

An embodiment provides that the modifying in accordance with the modification instruction comprises volume normalization of the speech sounds with respect to a reference value. The reference value can be an average volume level of the speech sounds of other patients, wherein the reference value is retrieved for instance from the memory unit before the normalization. This embodiment is advantageous in particular because it ensures that the user of the imaging system can perceive even comparatively soft-spoken patients.

In general, a trained machine learning model mimics cognitive functions that humans associate with other human minds. In particular, the machine learning model can, through training based on training data, be adapted to new circumstances and recognize and extrapolate patterns. Another term for “trained machine learning model” is “trained function”. The parameters of a machine learning model can be adapted in general through training. In particular, supervised, semi-supervised, unsupervised training methods, reinforcement learning and/or active learning can be used. Furthermore, representation learning (an alternative term is “feature learning”) can be used. In particular, the parameters of the machine learning models can be adapted iteratively by a plurality of training steps. In particular, a specific cost function can be minimized in the training. In particular, the backpropagation algorithm can be used in the training of a neural network. A machine learning model can include in particular a neural network, a support vector machine, a decision tree, and/or a Bayes network, and/or the machine learning model can be based on k-means clustering, Q-learning, genetic algorithms and/or association rules. In particular, a neural network may be a deep neural network, a convolutional neural network, or a deep convolutional neural network. In addition, a neural network can be an adversarial network, a deep adversarial network, and/or a generative adversarial network.

The use of convolutional neural networks allows very efficient processing of input images, for example, because a convolution based on different kernels can extract different image features, and therefore the relevant image features can be found in the training by adapting the weights of the convolution kernel. Moreover, by virtue of the weight-sharing in the convolution kernels, fewer parameters have to be trained, which prevents overfitting in the training phase and allows faster training or more layers in the network, thereby improving the performance of the network. This applies analogously to sounds.

According to one aspect, the machine learning model can comprise one or more residual networks (ResNet). In particular, a ResNet is an artificial neural network that has at least one skip connection, which jumps over at least one layer of the artificial neural network. In particular, a ResNet is a convolutional neural network that has one or more skip connections, which each jump over one or more convolutional layers. According to some examples, the ResNets can be represented as m-layer ResNets, where m is the number of layers in the corresponding architecture, and in some examples can have values of 34, 50, 101 or 152. According to some examples, each such m-layer ResNet can comprise (m−2)/² skip connections. A skip connection can be regarded as a bypass that forwards the output of a preceding layer directly over one or more skipped layers to a downstream layer. Instead of adapting a desired mapping directly, the skipped layers would then have to “balance out” a residual mapping, which regularizes the directly forwarded output.

The adapting of the residual mapping is computationally simpler to optimize than the direct mapping. Furthermore, it solves the vanishing/exploding gradient problem during optimization when training the machine learning model: if a skipped layer encounters such a problem, its contribution can be skipped by regularizing the directly forwarded output. The use of ResNets has the advantage that far deeper networks can be trained. In the context of the intended providing of classified system sounds, for example, this has the advantage of predicting classified system sounds more accurately, and thus helps to improve the assignment.

A generative adversarial model (with the acronym GA model) comprises a generative function and a discriminative function, where the generative function generates synthetic data, and the discriminative function discriminates between synthetic and real data. Through the training of the generative function and/or of the discriminative function, the generative function is configured to generate synthetic data that the discriminative function incorrectly classifies as real, while the discriminative function is configured to discriminate between real data and synthetic data generated by the generative function. In terms of game theory, a generative adversarial model can be interpreted as a zero-sum game. The training of the generative function and/or of the discriminative function is based in particular on minimizing a cost function.

By using a GA model, on the basis of a set of training data can be produced synthetic data that has the same properties as the training dataset. The training of the GA model can be based on unannotated data (unsupervised learning), which means that less effort is needed for training a GA model.

A recurrent machine learning model is in general a machine learning model, the output from which depends not only on the input value and the parameters adapted by the training process but also on a hidden state vector, which hidden state vector is based on previous input values used for the recurrent machine learning model. In particular, the recurrent machine learning model can comprise additional memory states or additional structures which contain time delays or comprise feedback loops.

In particular, the underlying structure of a recurrent machine learning model can be a neural network, which is called a recurrent neural network. Such a recurrent neural network can be described as an artificial neural network in which connections between nodes along a time series form a directed graph. In particular, a recurrent neural network can be interpreted as a directed acyclic graph. In particular, the recurrent neural network can be a finite impulse recurrent neural network or an infinite impulse recurrent neural network (where a finite impulse network can be unrolled and replaced with a strictly feedforward neural network, and an infinite impulse network cannot be unrolled and replaced by a strictly feedforward neural network).

In particular, the training of a recurrent neural network can be based on the BPTT algorithm (acronym for “backpropagation through time”), the RTRL algorithm (acronym for “real-time recurrent learning”) and/or on genetic algorithms.

By using a recurrent machine learning model, it is possible to use input data that comprises variable-length sequences. This means in particular that the method can be used not just for a fixed number of input datasets (and does not have to be trained differently for every other number of input datasets used as input), but can be used for any number of input datasets. This means that the entire set of training data, irrespective of the number of input datasets in different sequences, can be used in training, and that training data is not reduced to training data that equates to a certain number of successive input datasets.

A transformer network is a neural network architecture that in general comprises an encoder, a decoder or both an encoder and a decoder. In some cases, the encoder and/or decoder consist of a plurality of corresponding encoding layers and decoding layers. Within each encoding and decoding layer is preferably an attention mechanism. The attention mechanism, sometimes called self-attention, relates datapoints (such as words or pixels) within a group of datapoints to other datapoints within this group. The self-attention mechanism allows the model to examine, for example, a segment of sounds within a detected audio signal and to establish the relative meaning of other segments of sounds within this audio signal.

The encoder can be configured in particular to convert the input into a numerical representation. The numerical representation can comprise one vector per input token, for instance per sound. The encoder can be configured to implement an attention mechanism so that each vector of a token is affected by the other tokens in the input. In particular, the encoder can be configured such that the representations resolve the desired output of the transformer network.

The decoder can be configured in particular to convert an input into a sequence of output tokens. The decoder can be configured in particular to implement a masked self-attention mechanism so that each vector of a token is affected only by the other tokens at one end of a sequence. Furthermore, the decoder can be autoregressive, which means that interim results, for instance a previously predicted sequence of tokens, are fed back. In some examples, the input to the decoder is based on the output from the encoder or equals the output from the encoder. In addition, the transformer network can comprise a classifier module, which maps the output from the encoder or decoder onto a group of learned outputs, in particular from one, a plurality of, or all of the following steps:
capturing an audio input signal via at least one microphone, wherein the at least one microphone is set up and oriented so as to detect, and provide as the audio input signal, sounds of the patient and of the imaging system while the imaging examination is being conducted;
extracting speech sounds of the patient and system sounds of the imaging system from the audio input signal via a computer unit;
classifying the system sounds via the computer unit;
weighting, via the computer unit, the classified system sounds with at least two different weighting factors retrieved from a memory unit;
determining a modification instruction, which depends on the patient of the imaging examination, by analyzing the perceptibility of the extracted speech sounds via the computer unit;
modifying the speech sounds and/or the weighted system sounds in accordance with the modification instruction via the computer unit;
combining the possibly modified speech sounds and the possibly modified system sounds into an audio output signal via the computer unit.

In some examples, the training of a transformer model can be performed in two phases: a pre-training phase and a fine-tuning phase. In the pre-training phase, a transformer model can be trained from a large corpus of data in order to learn the underlying semantics of the problem. Such pre-trained transformer models are available for different languages. For certain applications described here, the fine-tuning can comprise further training of the transformer network using medical texts containing expert-annotated meanings and/or medical ontologies such as RADLEX and/or SNOMED. Thus in some examples, the transformer model can learn typical relationships and synonyms of medical expressions.

An advantage of transformer networks is that transformer networks can handle long-range relationships in input data efficiently by virtue of the attention mechanism. Furthermore, encoders used in transformer networks are capable of processing data in parallel, which saves computing resources in the inference. In addition, decoders of transformer networks, by virtue of the autoregression, are capable of producing a sequence of output tokens iteratively with great confidence.

The computer unit can comprise in particular program code means containing at least one trained machine learning model that is set up for the extracting, the classifying, the determining and/or the combining. The computer unit can comprise in particular the extraction program code means, the classification program code means, the weighting program code means, the determination program code means, the modification program code means and/or the combining program code means.

The term computer program product should be understood in the broad sense here. The computer program product can be a computer program or comprise a computer program. The computer program product comprises in particular the program code means that model the method steps according to one or more example embodiments. It is thereby possible to define and repeatedly perform the method according to one or more example embodiments, and to exercise control over disseminating the method according to one or more example embodiments. The computer program product is preferably configured such that the computing unit can use the computer program product to execute the method steps according to one or more example embodiments. The program code means can be loaded in particular into a memory of the computing unit, and typically can be executed by a processor of the computing unit with access to the memory. When the computer program product, in particular the program code means, is executed in the computing unit, typically all the embodiments according to one or more example embodiments of the described method can be implemented. The computer program product is stored, for example, on a physical, computer-readable medium and/or digitally as a data packet in a computer network. The computer program product can constitute the physical, computer-readable medium and/or the data packet in the computer network. Hence one or more example embodiments can also proceed from said physical computer-readable medium and/or from said data packet in the computer network. The physical, computer-readable medium can usually be connected directly to the computing unit, for instance by inserting the physical, computer-readable medium into a DVD drive or by plugging same into a USB port, whereby the computing unit can have access, in particular read access, to the physical, computer-readable medium. The data packet can preferably be retrieved from the computer network. The computer network can comprise the computing unit or be connected directly to the computing unit via a wide area network (WAN) connection and/or via a (wireless) local area network (WLAN or LAN) connection. For instance, the computer program product may be held digitally on a cloud server at a storage location of the computer network, and be transferred via the WAN via the Internet and/or via the WLAN or LAN to the computing unit, in particular by opening a download link that points to the storage location of the computer program product.

Features, advantages or alternative embodiments mentioned in the description of the device can also be applied to the method, and vice versa. In other words, claims relating to the method can be developed by features of the device, and vice versa. In particular, the device according to one or more example embodiments can be used in the method.

People of male, female or other gender identity are included regardless of the gender applied to a particular term.

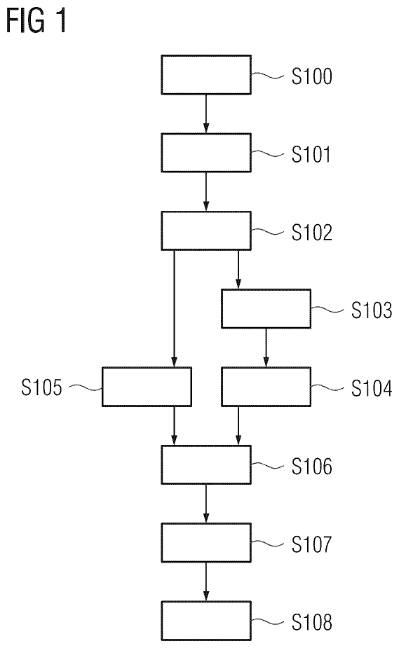

FIG. 1 shows a flow diagram of the method according to invention for conducting an imaging examination of a patient via a medical imaging system, in particular a CT or MRI or C-arm imaging system, containing the steps S100 to S108.

Method step S100 denotes starting the imaging examination via the imaging system.

Method step S101 denotes capturing an audio input signal via at least one microphone, wherein the at least one microphone is set up and oriented so as to detect, and provide as the audio input signal, sounds of the patient and of the imaging system while the imaging examination is being conducted.

Method step S102 denotes extracting speech sounds of the patient and system sounds of the imaging system from the audio input signal via a computer unit.

Method step S103 denotes classifying the system sounds via the computer unit. The system sounds comprise sounds from at least two of the following sound classes, wherein the system sounds are divided during classification into the at least two sound classes:
unwanted background sounds;
patient couch, in particular scan-specific movement of the support part of the patient couch and/or travel of the entire patient couch;
MRI coil, in particular gradient coil;
coolant pump, in particular helium pump;
rotating part of the CT gantry;
displaceable part of the CT gantry;
X-ray source;
contrast-agent injection pump;
patient instruction;
C-arm;
vital-parameter sensor, in particular patient monitoring system; and/or
external equipment.

Method step S104 denotes weighting, via the computer unit, the classified system sounds with at least two different weighting factors retrieved from a memory unit.

Method step S105 denotes determining a modification instruction, which depends on the patient of the imaging examination, by analyzing the perceptibility of the extracted speech sounds via the computer unit.

Method S106 denotes modifying the speech sounds and/or the weighted system sounds in accordance with the modification instruction via the computer unit.

Method S107 denotes combining the possibly modified speech sounds and the possibly modified system sounds into an audio output signal via the computer unit.

Method step S108 denotes providing, via a reproduction unit, to a user of the imaging system for monitoring the imaging system the combined audio output signal while the imaging examination is being conducted.

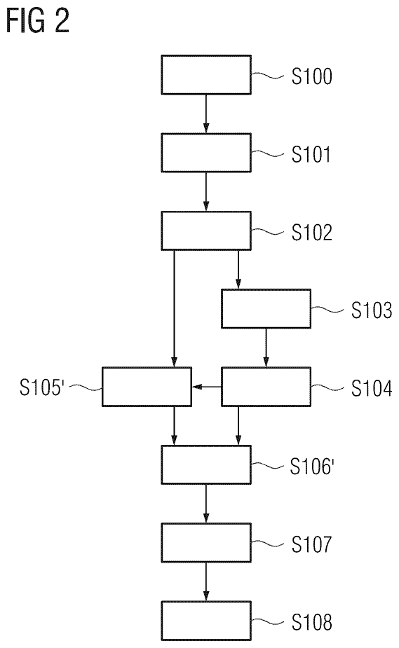

FIG. 2 shows a flow diagram of the first exemplary embodiment of the method.

Method step S105′ denotes determining a modification instruction, which depends on the patient of the imaging examination, by analyzing the perceptibility of the extracted speech sounds via the computer unit. The determining of the modification instruction is performed depending on the time-resolved weighting factor of the classified system sounds. The determining of the modification instruction further comprises the analyzing of the perceptibility of the speech sounds in comparison with the weighted system sounds depending on a human recipient, in particular the user of the imaging system. The modification instruction comprises a shift in the time of playback of at least one segment of the speech sounds. The shift equals 20 s maximum, in particular less than 2 s or less than 1 s, but more than 0 s.

Method S106′ denotes modifying the speech sounds and/or the weighted system sounds in accordance with the modification instruction via the computer unit, wherein, before being combined, the speech sounds and/or the system sounds are modified via an adjustment to the phase and/or frequency and/or amplitude and/or time of playback. The time of playback is modified in accordance with the modification instruction such that a time overlap with a segment of the system sounds is reduced. The modifying in accordance with the modification instruction comprises volume normalization of the speech sounds with respect to a reference value, wherein the reference value is an average volume level of the speech sounds of other patients, wherein the reference value is retrieved before the normalization.

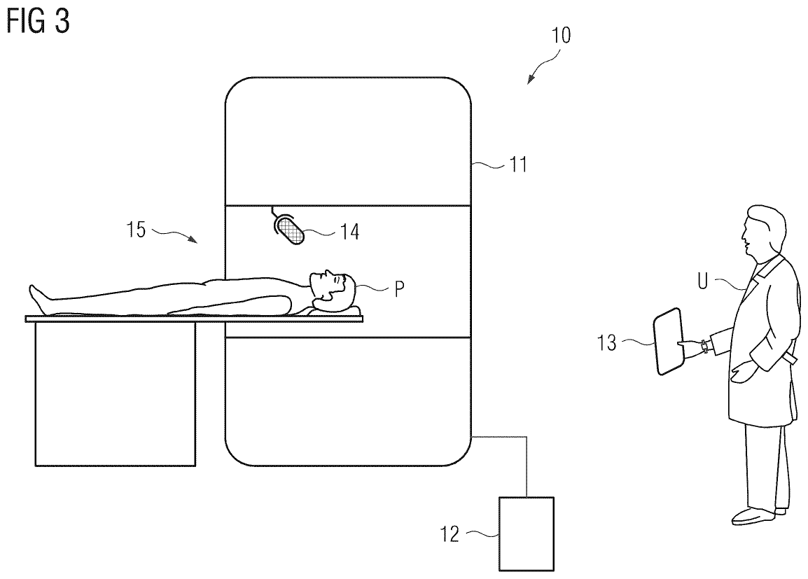

FIG. 3 shows a schematic block diagram of the imaging system 10 according to one or more example embodiments for performing the method according to one or more example embodiments.

The imaging system 10 has an imaging unit 11, the computer unit 12, the reproduction unit 13 for the user U and the at least one microphone 14. The imaging system 10 can be in particular a CT or MRI or C-arm imaging system. A CT imaging system is shown by way of example in FIG. 3. The imaging system 10 also has an examination region 15, in which the patient P is located while the imaging examination is being conducted. In FIG. 3, the at least one microphone 14 is oriented onto the examination region 15 containing the patient P. The reproduction unit 13 is connected wirelessly to the computer unit 12.

Although the invention has been illustrated and described in detail using the preferred exemplary embodiments, the invention is not limited by the disclosed examples, and a person skilled in the art can derive other variations therefrom that are still covered by the scope of protection of the invention.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,“ ”connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a Graphics Processing Unit (GPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility (also referred to as an data processing system)or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particular manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

1. A method for conducting an imaging examination of a patient via a medical imaging system, the method comprising:
starting the imaging examination via the imaging system;
capturing an audio input signal via at least one microphone, the at least one microphone is positioned to detect and provide sounds of the patient and of the imaging system as the audio input signal during the imaging examination;
extracting speech sounds of the patient and system sounds of the imaging system from the audio input signal via a computer unit;
classifying the system sounds via the computer unit;
weighting, via the computer unit, the classified system sounds with at least two different weighting factors retrieved from a memory unit;
determining a modification instruction based on the patient of the imaging examination by analyzing an perceptibility of the extracted speech sounds via the computer unit;
modifying at least one of the speech sounds or the weighted system sounds in accordance with the modification instruction via the computer unit;
combining the modified sounds into an audio output signal via the computer unit; and
providing, via a reproduction unit, to a user of the imaging system the combined audio output signal during the imaging examination for monitoring the imaging examination.

2. The method of claim 1, wherein the system sounds comprise sounds from at least two of the following sound classes, and wherein the system sounds are divided during classification into the at least two sound classes:
unwanted background sounds;
patient couch;
MRI coil;
coolant pump;
rotating part of a CT gantry;
displaceable part of the CT gantry;
X-ray source;
contrast-agent injection pump;
patient instruction;
C-arm;
vital-parameter sensor; or
external equipment.

3. The method of claim 2, wherein time segments of the sounds are assigned time-resolved weighting factors in accordance with the at least two sound classes.

4. The method of claim 1, wherein the modifying includes a shift in a time of playback of at least one segment of the speech sounds.

5. The method of claim 4, wherein the shift is less than or equal to 20 s maximum and greater than 0 s.

6. The method of claim 4, wherein the time of playback is modified in accordance with the modifying such that a time overlap with a segment of the system sounds is reduced.

7. The method of claim 3, wherein the determining of the modification instruction is performed depending on the time-resolved weighting factor of the classified system sounds.

8. The method of claim 1, wherein the analyzing includes analyzing the perceptibility of the speech sounds in comparison with the weighted system sounds based on a human recipient.

9. The method of claim 8, wherein the user of the imaging system is the recipient.

10. The method of claim 1, wherein the modifying comprises a volume normalization of the speech sounds with respect to a reference value.

11. The method of claim 10, wherein the reference value is an average volume level of the speech sounds of other patients, wherein the reference value is retrieved before the normalization.

12. The method of claim 1, wherein, before being combined, the at least one of the speech sounds or the system sounds are modified via an adjustment to at least one of a phase, a frequency, an amplitude, or a time of playback.

13. An imaging system configured to perform the method of claim 1, the system comprising:
an imaging unit;
the computer unit;
the reproduction unit; and
the at least one microphone.

14. The imaging system of claim 13, wherein the computer unit is configured to execute at least one trained machine learning model for at least one of the extracting, the classifying, the determining or the combining.

15. A non-transitory computer readable medium comprising instructions which, when executed by a computing unit, cause said computing unit to perform the method of claim 1.

16. The method of claim 2, wherein
the patient couch class is at least one of a scan-specific movement of a support part of the patient couch or travel of all of the patient couch,
the MRI coil is a gradient coil, and
the coolant pump is a helium pump.

17. The method of claim 5, wherein the shift is less than 2 s.

18. The method of claim 2, wherein the modifying includes a shift in a time of playback of at least one segment of the speech sounds.

19. The method of claim 4, wherein the determining of the modification instruction is performed depending on the time-resolved weighting factor of the classified system sounds.

20. The method of claim 2, wherein the analyzing includes analyzing the perceptibility of the speech sounds in comparison with the weighted system sounds based on a human recipient.
